(19) United States
(12) Patent Application Publication
(10) Pub. No.: US 2005/0130287 A1
(43) Pub. Date: Jun. 16, 2005

(54) Purifying process of soluble proteins of the l.obliqua bristles through prothrombin activation: process for a partial determination of the amino acids sequence of the prothrombin activator; process for determining the prothrombin activation of fraction II, n-terminal and internal fragments sequences

(76) Inventors: Ana Chudzinski-Tavassi, Sao Paulo (BR); Cleyson Reis, Sao Paulo (BR)

Correspondence Address: BIRCH STEWART KOLASCH & BIRCH, PO BOX 747, FALLS CHURCH, VA, 22040-0747, US

(21) Appl. No.: 10/501,238

(22) PCT Filed: Jan. 29, 2003
(86) PCT No.: PCT/BR03/00012

(30) Foreign Application Priority Data
Jan. 31, 2002 (BR) PI02002698

(51) Int. Cl. C12N 9/20; C12N 9/64

(52) U.S. Cl. 435/226; 435/198

(57) ABSTRACT

The herein invention refers to a purifying process of soluble proteins of the L. obliqua bristles through prothrombin activation; a partial deterrination of the amino acids sequence of the prothrombin activator; a process for determining the fraction II of the prothrombin activation as well as the N-terminal sequence and the sequence of internal fragments of the prothrombin activator fraction, the prothrombin activator and the utilization of the prothrombin activator through the homogenization of the L. obliqua bristles. The herein invention has shown that only one component of the Lonomia obliqua venom, the Lopap, causes the hemorrhagic syndrome directly by activating prothrombin and, therefore, a patient should be conducted to a therapy when in contact with the Lonomia obliqua venom. According to the herein invention, Lopap is a new prothrombin activator, showino to be a quite important factor responsible for consumption coagulopathy, found in patients exposed to the venom of the L. obliqua caterpillar. In low doses of purified protein, due to its capacity of activating prothrombin and generating thrombin, it is possible, in controlled conditions, to withdraw fibrinogen from circulation, transforming it in fibrin microthrombs. The decrease on the concentration of plasmatic fibrinogen promotes the increasing of blood coagulation time and therefore it will avoid acute vascular thrombosis. Since protein does not present proteolytic activity, it could maintain the coagulating capacity of the fibrinogen not consumed in the process. The fibrinogen plasmatic concentration would decrease, however there would not be predisposition for hemorrhagic state. Besides that, it could be used to produce diagnosis KITS for detecting dysprothrombinemias.

### STATEMENT OF THE OBJECT OF THE INVENTION

The herein invention refers to a purifying process of soluble proteins of the L. obliqua bristles through prothrombin activation; a process for a partial determination of the amino acids sequence of the prothrombin activator; to the process for determining the prothrombin activation of fraction II, N-Terminal and internal fragments sequences of the prothrombin activator fraction, as well as the prothrombin activator and the utilization of the prothrombin activator.

### BACKGROUND OF THE INVENTION

Prothrombin is a plasmatic protein, vitamin K dependent related to blood coagulation. The activation of prothrombin is speeded up through the prothrombinase complex, which is composed by Factor Xa, Factor Va, phospholipides and calcium ions and is obtained through the cleavage (in sequence) when linking between two peptides of the prothrombin's molecule (Mann K G. Prothrombin and Thrombin. In: Colman R W, Marder V J, Salzman E W, Hirsh J eds. Haemostasis and Thrombosis. Basic Principles and Clinical Practice. Philadelphia: J. B. Lippincott; 1994. P 184-99).

The first cleavage occurs between bounds Arg320 and Ile 321, and this hydrolysis comes to be an intermediate activator—meizothrombin. Its second cleavage occurs between bounds Arg271 and Thr272 or amino acids residues, and brings out fragments 1, 2 and the serine protease α-thrombin (Mann K G. Prothrombin and Thrombin. In: Colman R W, Marder V J, Salzman E W, Hirsh J eds. Haemostasis and Thrombosis. Basic Principles and Clinical Practice. Philadelphia: J. B. Lippincott; 1994. P .184-99).

When phospholipides are not present, prethrombin can be activated by physiological concentrations of factor Xa, however, its activation speed is 5 grades lower when compared to its activation through prothrombinase complex (Mann K G. Membrane-bound enzyme complexes in blood coagulation. Prog. Hemost Thromb. 1984; 7:1-23.), and its activating mechanism occurs through prethrombin formation (Mann K G. Membrane-bound enzyme complexes in blood coagulation. Prog. Hemost Thromb. 1984; 7:1-23.) instead of meizothrombin (Heldebrandt C M, Butkowski R J, Bajaj S P, Mann K G. The activation of prothrombin. H. Partial reactions, physical and chemical characterization of the intermediates of activation. J Biol. Chem. 1973; 248: 7149-63).

α-thrombin is the serine protease that transforms fibrinogen into fibrin, activates factors V, VIII, and XIII, and. aggregates platelets (Mann K G, Downing M R. Thrombin generation. In: Lundblad R L, Fenton J W, Mann K G, Eds. Chemistry and Biology of Thrombin. Ann Arbor Science; 1977. Pp. 11-21; Lundblad R L, Kingdon H S, Mann K G. Thrombin. Methods Enzymol. 1976; 45:156-76).

Many venomous snakes have procoagulant proteins, which can activate zymogens, related to blood coagulation.

Since the mechanisms of which venomous enzymes activate coagulation factors in a different way than of those found in mammals, venom activators could be adding information concerning the activation mechanisms of blood coagulation. The prothrombin activators from venom are classified as Type 1 (e.g. ecarina), Type 2 (e.g. Notechis scutatus activator), 3 (e.g. Oxyuranus scutellatus), and 4. (e.g. Agkistrodon acutus activator) depending on its interaction ith the components of the prothrombinase complex (Rosing J. Tans G. Inventory of exogenous prothrombin activators. Thromb. Haemost. 1991; 65 (5): 627-30).

Type 1 activators do not depend on the prothrombinase complex components while those of Type 2, depend on phospholipides, Ca ²⁺and Factor Va, of Type 3 depend on phospholipides and Ca ²⁺Activators of Type 4 may or may not need the prothrombinasis complex components and can cleave peptide bounds in prothrombin without converting it into catalytic activity products (e.g. thrombin or meizothrombin).

Activators of Type 4 and thrombin hydrolyzes prothrombin at the same way (Argl55-Ser156 and Arg284-Thr285), forming similar or identical fragments to prethrombin 1 and prethrombin 2 (Rosing J, Tans G. Structural and functional properties of snake venom prothroinbin activators. Toxicon. 1992; 30: 1515-27.)

In the Lonomia achelous hemolymph, two types of prothrombin activators were described. One of them is able to directly activate prothrombin, independently of the prothrombinase complex; (the Factor V, calcium ions, and phospholipides) (GUERRERO BAG, Arocha-Pinango stimulate the other Activation of human prothrombin by the venom of Lonomia achelous (Cramer) caterpillars. Thrombos. Res. 1992;66:169-77).

A procoagulant activity was described when the crude extract of L. obliqua bristles was analyzed, by the activation of prothrombin and Factor X (Kelen ELA. Duarte A. C, Tomy S C, Sano-Martins IS, Castro SCB, Guerrero B, Arocha-Pinãngo CL. Acquired haemrorrhagic syndrome from contact with a caterpillar (Lonomia obliqua Walker 1855, Saturniidae). Toxicon 1996: 34:146, Donato J L, Moreno R A, Hyslop S. Duarte A C, Antunes E, Le Bonniec B F, Rendu F, Nucci G. Lonomia obliqua caterpillar spicules trigger human blood coagulation via activation of factor X and prothrombin. Thromb. Haemost. 1998; 79: 539-42).

The venom of Lonomia obliqua causes a severe consumption coagulopathy, which can result in an hemorrhagic syndrome. The crude bristles extract presents a procoagulant activity via the factor X and prothrombin activation.

Since 1989, this hemorrhagic syndrome caused by the contact with the Lonomia obliqua caterpillar has become epidemic in Brazil and fatal cases, due to renal damages and cerebral hemorrhage have been described. Those damages affect coagulation mechanism, resulting in a drastic reduction of fibrinogen, as well as reduction of factors V and XIII. It can also be noticed a decrease of the α-antiplasmine plasminogen levels and of the C protein activity, a natural coagulation inhibitor. These data indicate a consumption coagulopathy via fibrinogen depletion.

The damage symptoms caused by the contact with the Lonomia obliqua caterpillar are urticant dermatitis, ecchymosis and hematomas (as spontaneous reaction or as results of traumas), hemorrhage at mucous cavities (gingival, nasal hemorrhage), hematuria, recent wounds bleeding, and abdominal, pulmonary, glandular and cerebral hemorrhages. Fatal cases have been related to renal damages and cerebral hemorrhages.

Previous studies concerning to damages caused by the contact with the L. achelous in Venezuela suggested that in such cases, the hemorrhagic syndrome could be explained as a severe fibrinolytic syndrome, which was associated to a disseminated intravascular coagulation. Although the clinical symptoms of envenoming by contacts with Lonomia achelous and Lonomia obliqua are quite similar, researchers of this herein invention have demonstrated and suggested a different interpretation for this last mentioned, based on results from fulfilled studies in laboratories. Thrombin formation has shown to be the main molecular mechanism of the hemorrhagic syndrome caused by the contact with the Lonomia obliqua.

More specifically, in the ten last years, literature has been showing the increase of human hemorrhagic syndrome cases in the South of Brazil, caused by the contact with the Lonomia obliqua caterpillar. Its venom causes a severe consumption coagulopathy, which can result in hemorrhagic syndrome.

The herein invention is based on the statement that a crude extract prepared from the Lonomia obliqua bristles activates both the prothrombin and Factor X. In accidental envenoming, there are alterations in coagulation and in fibrinolytic factors.

Lopap (Lonomia obliqua prothrombin activator protease) is a serine protease of 69 kDa isolated from the Lonomia obliqua caterpillar bristles extract, and its activity is increased in presence of Ca ²⁺and is able to convert prothrombin into thrombin on a dose-dependent manner. Its mechanism of action is similar to that of Factor Xa, generating fragments of prethrombin independently of the prothrombinase complex components. Lopap hydrolyses a fluorogenic substrate based on the prothrombin sequence at the same peptide bound as the thrombin.

This herein invention also starts from verifying the biological characterization of the prothrombin activator serine protease isolated from the crude extract of the Lonomia obliqua bristles, reproducing the whole venom effects in blood coagulation and thrombin formation in rats.

According to this invention, purified Lopap can be obtained from the crude extract of the Lonomia obliqua bristles in PBS, the prothrombin activator was purified by gel filtration, and two chromatography stages in reverse phase. The activity of prothrombin activator was monitored using the chromogenic substrate S-2238 and cleaved by the thrombin.

This herein invention comes to state that only one component of the Lonomia obliqua venom, Lopap, can directly cause the hemorrhagic syndrome via prothrombin activation, therefore a therapy should be provided in case of accidental contact with Lonomia obliqua.

When evaluating the effects of the Lopap injection in rats, on coagulation parameters, the reaction of the microcirculatory blood vessels and the damages in different body organs when doses of 100 μg/kg were injected and the effect monitored for 1 hour, has shown that blood becomes unclotable. Platelet count is reduced in about 40% and inducing of the platelet aggregation via collagen at the whole blood was completely annulled. Although in presence of high concentration of toxins, the number of erythrocytes and of leukocytes at the whole blood was not altered, however, intense venous occlusion and hemorrhagic areas were observed. The generation of intravascular thrombin can explain the decrease of the platelet count and the platelet hypoaggregation after the Lopap injection in rats, being the thrombin the main and most active platelet agonist.

Observing the microcirculatory system 5 minutes after administering Lopap injection, fibrin clots in postcapillary vessels can be observed. Prominent alterations occur 1 h after this administration, when occlusion in some of the blood vessels and intense hemorrhagic areas were verified. This phenomenon may be connected to the hematomas observed in the majority of the human patients who were exposed to such venom. Histological analyses in several organs on experiment animals were conducted 1 hour after administering the Lopap injection. Alterations were found only on pulmonary and renal tissues, being the last mentioned the most significant since hemorrhagic and necrotic areas could be verified. Patients classified as mild or severe envenoming usually present hematuria and sometimes renal deficiency; sometimes fatally. Renal lesions found in experiment rats could be caused by the hemorrhage and/or by the fibrin deposit in the glomerulus. It may be true that during a longer envenoming time, microthrombs and blood congestion signals in other organs, including the central nervous system can be verified.

Based on these statements the herein invention describes Lopap as a new prothrombin activator, a very important factor responsible for the main symptoms found in human patients envenomed by the Lonomia obliqua caterpillar.

For evaluating whether one or more prothrombin activators of the caterpillar toxin is involved, soluble proteins of the Lonomia obliqua bristles were purified by gel-filtration and on reverse-phase of high performance liquid chromatography (HPLC). Prothrombin activation was monitored using prothrombin and the specific chromogenic substrate for thrombin S-2238, from Chromogenix. The products of the prothrombin hydrolysis were also identified by SDS-PAGE. A protein of 69 kDa come out as a serine protease. activated by calcium ions, directly converting prothrombin into thrombin and it might be included in group 1 of the prothrombin activators. “Lonomia obliqua Prothrombin Activator Protease” (Lopap) was purified until homogeneity and its amino acids sequence neither present homology with other prothrombin activators nor with any other serine protease.

Experiments “in vivo” have shown that when the purified protein is injected in rats, the same effects are obtained as those of crude extract of bristles, therefore unclotable blood is verified in a dose-dependent manner. This has corroborated by observing the microcirculatory system of the cremaster muscle after injecting the protein using the intravital microscopy technique. The data obtained have shown that the Lopap infusion produces.an intravascular coagulation and thrombosis in post-capillary vessels, what frequently contributes to organ damages. Lopap is surely the main factor causing the consumption coagulopathy after accidental contact with the L. obliqua bristles.

A main aspect of the herein invention is related to the Soluble Proteins Purifying Process of the L. obliqua bristles with prothrombin activator activity. It is performed by the homogenization of the L. obliqua bristles in phosphate-buffered saline (PBS), on pH between 7.4 and 8.0 followed by centrifugation of 2500×g on temperature ranging from 4° to 10° C. during 30 to 60 minutes in order to obtain a crude extract. Then, purification of the prothrombin activator from the crude extract is performed from 50 to 200 mg of the whole protein in 2 to 10 ml of crude extract. It is reached by gel filtration chromatography in Sephadex G-75 resin, through elution in 20 to 50 mM Tris-HCL buffer containing NaCl 50 to 100 mM and benzamidine 2 to 5 mM on pH level from 7.4 to 8.0 with flow of 1, 0 ml/h. Then, fractions can be collected from 1 to 3 ml and the protein profile can be monitored by UV absorbency in 280 nm.

Prothrombin is activated in material obtained in protein peaks using the S-2238 colorimeter substrate specific for thrombin, in order to obtain peak PII, which shall contain prothrombin activator action. The active peak is exposed to a reverse-phase chromatography through the C4 column in HPLC analytical system. As following solvents were used: A: 0,1% TFA in water (balanced) and B: solvent A and acetonitrile in a proportion of 1:9 (elution) that is, solvent B: 100 ml of solvent A with adding of 900 ml of acetonitrile. A gradient of 35-50% of solvent B is used for 30 minutes and the protein detection is monitored using 214 or 280 nm in an UV detecting monitor. After that, fractions of 0.5-1.0 ml are collected and immediately lyophilized for eliminating acetonitrile.

The next procedure is to solubilize the lyophilized samples in Tris-HCl from 20 to 50 mM buffer containing NaCl from 50 to 100 mM on pH from 7.4 to 8.0. The prothrombin activator activity on these fractions using what was obtained in the protein peaks was measured by the S-2238 chromogenic substrate specific for thrombin.

The active peak is obtained in eluted fractions between 42 and 44% of B solvent.

New chromatography of the active fraction by reverse phase chromatography through column C4 in an HPLC analytical system. The following solvents were used: A: 0,1% TFA in water (balanced) and B: solvent A and acetonitrile in a proportion of 1:9 (elution), that is, solvent B: 100 ml of solvent A adding 900 ml of acetonitrile using a linear gradient between 20-80% of solvent B, during 20 minutes. The protein detection using absorbency 214 or 280 nm in UV monitor is performed. Fractions of 0.5-1.0 ml are collected and lyophilized immediately in order to eliminate acetonitrile. Lyophilized samples were again suspended in Tris-HCL 20 to 50 mM buffer containing NaCl 50 to 100 mM in a pH from 7.4 to 8.0. The prothrombin activator activity of the fractions is measured using what was obtained in the protein peaks through the S-2238 chromogenic substrate specific for thrombin. It can be observed that the active peak is in the fractions eluted between 42 and 44% of the B solvent.

The purified material can be submitted to electrophoresis in polyacrilamide gel containing SDS for homogeneity evaluation. This gel may be stained using coomassie brilliant blue.

The measure of the final protein concentration can be evaluated through protein measure using calorimetry methods or by Absorbency in 280 nm.

Merck-Hitachi (model D-2500) and the monitor Shimadzu UV (SPD-6AV model) produce the HPLC analytic system applied.

In the process of the herein invention, the following solvents were used for elution:

solvent A: 0,1% TFA in water

Solvent B: solvent A and acetonitrile in a proportion of 1:9. or even else, 100 ml of solvent A with adding of 900 ml of acetonitrile.

For fulfilling purification in HPLC a gradient of 35-50% of solvent B is used.

Another invention is related Lo the Process for the partial determination of the amino acids sequence of the prothrombin activator. There, 500 to 1000 pM of the purified protein were degrade by 10 pmol of trypsin in 100 M Tris-HCl, on pH 8.0 containing 0.02% of CaCl₂ during 18 hours at 37° C. ending the reaction with 15% (v/v) of formic acid. In this process, the fragments obtained are separated in HPLC using a C4 column, elution solvents in a proportion of 0,1% of TFA in water (solvent A) and acetonitrile: solvent A (9:1) (solvent B). For fragments isolation separated by HPLC, a gradient of 0-100% of solvent B was used with a flow of 1.0 ml/min during 30min.

According to the process of the herein invention a sequence of four internal peptides and of the N-terminal were determined. The N-terminal portion contains 46 amino acids residues (DVVIDGACPDMKAVSKFDMNAYQGTWYEIKKFPVANEANGDCGSVE) and the internal peptide fragments are:

| | |
|---|---|
| Fragment I | (KSHVYTVPFGA); |
| Fragment II | (KSNQHRVNIWILSRTK |
| Fragment III | (VRAGHVE) |
| Fragment IV | (FDQSKFVETDFSEKACFF). |

The sequence obtained corresponds to about 15% of the whole protein and molecular mass of 69KDa.

Another invention is related to the process for determining the reaction of the prothrombin activator of fraction II. It comprehends the pre-incubate 15 to 300 nM of the purified fraction during 10 minutes at 37° C. with 90 pM of prothrombin and 5 mM of CaCl₂ for final volume of 500 μL of 50 mM Tris-HCl, 100 mM of NaCl, pH 8,0 as well as 150 mM of imidazol. It is added 40 μM of the chromogenic substrate S-2238 (H-D-phenylalanyl-L-pipecolyl-L-arginine-p-nitroaniline dihydrochloride) to the incubation mixture and evaluated by spectrometry in absorbency of 405 nm during 10 minutes the hydrolysis of the chromogenic substrate.

This invention is also related to the N-terminal sequence and the Sequence of internal fragments of the prothrombin activator fraction characterized by containing the N-terminal portion with 46 amino acids residues (DVVIDGACPDMKAVSKFDMNAYQGTWYEIKKFPVANEANGDCGSVE). The fragments of internal peptides are Fragment I (KSHVYTVPFGA); Fragment II (KSNQHRVNIWILSRTK); Fragment III (VRAGHVE) and Fragment IV (FDQSKFVETDFSEKACFF) resulting in a sequence that corresponds to about 15% of the whole protein and molecular mass of 69 Kda.

Another part of the invention is related to the prothrombin activator containing the following structure: The purified protein is characterized as a serine protease which hydrolyzes the prothrombin generating Fragments 1, 2 and thrombin.

Lastly, the invention aims to be using the prothrombin activator as a dysfibrinogening element in phrothrombotic states.

In low doses of purified protein, due to its capacity of activating prothrombin and generating thrombin, it is possible, in controlled conditions, to withdraw fibrinogen from circulation, transforming it in fibrin microthrombs. The decrease on the concentration of the plasmatic fibrinogen promotes the increasing of the coagulation time and therefore it will refrain acute vascular thrombosis.

Since protein does not present proteolytic activity, it could maintain the coagulation capacity of the fibrinogen not consumed in the process. This way the fibrinogen plasmatic concentration would decrease, however there would not be predisposition for hemorrhagic state. Besides that, it could be used to produce diagnosis KITS for detecting the plasmatic prothrombin.

Manner and Process of Making and Using it Reagents:

E-64 (trans-epoxysuccinil-L-leucilamide- (4-guanidine-butane)-prothrombin, EDTA (etilene-diaminotetraacetic acid), PMSF (phenylmethylsulphonil fluoride), NPGB (p-Nitrophenyl-p″-guanidinebenzoate) and trypsin were obtained from Sigma; S-2238 (H-D-phenylalanyl-L-pipecolyl-L-arginine-p-nitroaniline dihydrochloride) and S-2765 (N-α-benzyloxycarbonyl-D-arginyil-L-glycil-L-arginine-p-nitroanilide-dihydrochloride) were obtained from Chromogenix.

All the other reagents used in this invention were from the best available suppliers of the market. Sephadex G-75 resin was provided by Pharmacia, the C₄ (5 μm, 4., 6×250 mm) column by J. T. Baker, while column C ₁₈ (μBondapack 10 μm; 22,5 mm×250 mm) was provided by Millipore Corp. The fluorescent peptide substrate Abz-YQTFFNPRTFGSQ-EDDnp. (Abz=ortho-aminobenzoic acid; EDDnp=N-[2,4-dinitrophenyl] ethylenediamine), of which sequence is based on the prothrombin sequence, was made into synthetic at the Biophysics Department of the “Universidade Federal de São Paulo” (University of Sao Paulo), Brazil, in accordance with the procedures previously described.

The reference examples presented as follows will help better describing the herein invention.

However, these reference procedures and data refer merely to some categories of concrete evidences of the herein invention and should not be limiting its utilization.

### DETAILED DESCRIPTION OF THE INVENTION

### Description 1

Purification of Soluble Proteins of the L. Obliqua Bristles via Prothrobin Activation:

L. obliqua bristles were homogenized in phosphate-buffered saline (PBS), pH 7.4-8.0, centrifuged at 4° to 10° C. by 2500×g from 30 to 60 minutes obtaining a crude extract, which presented the prothrombin activator activity. The prothrombin activator was purified from 50 to 200 mg of whole protein from 2 to 10 ml of crude extract by gel-filtration chromatography in Sephadex G-75 resin. It was eluted in 20 to 50 mM Tris-HCl buffer containing NaCl 50 to 100 mM and benzamidine 2 to 5 mM, pH 7.4 to 8.0 with flow of 1,0 ml/h. Fractions from 1 to 3 ml were collected and the protein profile monitored by UV absorbency in 280 nm. Prothrombin was activated using the protein peaks obtained and the S-2238 colorimetric substrate, specific for thrombin.

Peak PII was obtained, which should contain the prothrombin activator, and it is submitted to one reverse-phase chromatography in C4 column using HPLC analytic system. As solvents were used: A: 0,1% TFA in water (balanced) and B: solvent A and acetonitrile in a proportion of 1:9 (elution). Then proceeding the protein detection of 214 to 280 nm in UV monitor and collecting fractions of 0.5-1.0 ml. Then they were immediately lyophilized for eliminating acetonitrile and suspended again in 20 to 50 mM Tris-HCl buffer containing 50 to 100 mM NaCl pH 7.4 to 8.0. This is conducted for. checking the prothrombin activator activity of the fractions eluted between 42% and 44% of solvent B. The active fraction is submitted again to a chromatography using a gradient between 20-80% of solvent B, during 20 minutes.

The purified material may be submitted to an electrophoresis in polyacrilamide gel containing SDS for homogeneity evaluation. This gel could be stained by Coomassie brilliant blue.

The dosage of the final protein can be evaluated by protein assay using calorimetric methods or by Absorbency in 280 nm.

### Description 2

Purification of Soluble Proteins of the L. Obliqua Bristles via Prothrobin Activation.

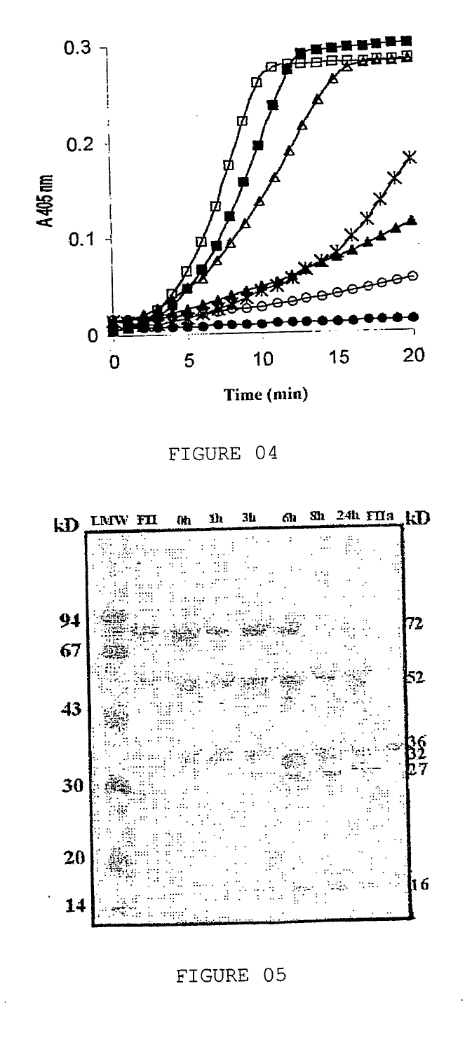

The L. obliqua caterpillars were anesthetized in CO₂ environment and their bristles were removed and stored in ice. The crude extract was obtained from 9.9 g of bristles homogenized in PBS, pH 7,4 and centrifuged by 2500 g at 4° C. during 10 minutes. The prothrombin activator was purified from the crude extract (103,5 mg in 12,0 ml) through gel-filtration chromatography (column: 100×1, 8 cm Sephadex G-75). It is eluted using 50 mM Tris-HCl buffer, containing 100 mM NaCl, 5 mM benzamidine, pH 8,0, with flow of 15 ml/h. Fractions of 2,0 ml were collected and the chromatography protein profile was monitored via UV absorbency in 280 nm. The prothrombin activation was verified using. the calorimetric substrate specific for thrombin (PII peak, protein: 5,68 mg). Active protein was submitted to a reverse-phase chromatography using column C₄ in HPLC analytic system by Merck-Hitachi (model D-2500), and a UV monitor by Shimadzu UV (model SPD-6AV) for protein detection in 214 nm. Elution solvents were TFA 0,1% in H₂O (solvent A) and acetonitrile: solvent A (9:1) (solvent B). Purification in HPLC was performed using a gradient of 35-50% of solvent B with flow of 1,0 ml/min during 30 minutes. The collected peaks were immediately lyophilized. The protein peak that presented prothrombin activation activity was suspended again in 50 mM Tris-HCl buffer. It contained 100 mM NaCl, pH 8,0, and submitted to a new chromatography in a gradient of 20-80% using solvent B, flow of 1,0 ml/min during 20 min, in the same column and conditions described above. The only peak obtained after the second chromatography at HPLC (PII-4R2) was collected and analyzed on SDS-PAGE. An aliquot of purified Lopap submitted to dialysis against 10 mM EDTA was used in the experiments described in FIG. 4.

The protein homogeneity was analyzed through SDS-PAGE using polyacrilamide gel 10% (p/v) stained by Coomassie Brilliant Blue R-250. The protein concentrations were determined in accordance with the method previously described and through absorbency in 280 nm. The activating capacity of the Lopap (300 nM) was tested in different concentrations of acetonitrile and after the lyophilization.

### Description 3

Partial Determination of the Amino Acids Sequence of the Prothrombin Activator:

Purified protein 500-1000 pM were degraded with 10 pmol of trypsin in 100 mM Tris-HCl, pH 8.0 containing 0.02% of CaCl₂ during 18 hours at 37° C. stopping the reaction with 15% (v/v) of formic acid.

Fragments isolation were obtained through HPLC in the C4 column eluted with solvents 0,1% of TFA in water (solvent A) and acetonitrile: solvent A (9:1) (solvent B)

It was used a gradient of 0-100% of solvent B with flow of 1.0 ml/min during 30min for the HPLC separation.

### Description 4

Partial Determination of the Amino Acids Sequence of the Lopap:

The purified Lopap (500 pM) was submitted to degradation through trypsin (10 pmol) in 100 mM Tris-HCl buffer, pH 8,0 containing 0,02% CaCl₂ during 18 h at 37° C. The reaction was interrupted using formic acid 15% (v/v). The fragments obtained were separated through HPLC using a C₄ column and the elution solvents were TFA 0,1% in H₂O (solvent A), and acetonitrile: solvent A (9:1) with solvent B.

For fragments separation in HPLC a gradient of 0-100% of solvent B was used with a flow of 1,0 ml/min during 30 min. The sequence of three internal peptides and of the N-terminal was determined through. the equipment from Applied BioSystem that performs the reactions of Edman (17) degradation. The data bank Swiss Protein DataBase was utilized to verify the homology of Lopap primary structure.

### Description 5

Prothrombin Activator Activity:

The capacity of Lopap activating prothrombin. was indirectly determined through the thrombin formation assay generated by the prothrombin, using the chromogenic S-2238 substrate. The prothrombin activation of the bristles extract, of the partially purified fractions and of the purified Lopap (15 to 300 nM) was evaluated after pre-incubation during 10 minutes at 37° C. with prothrombin (90 pM), using 5 mM of CaCl₂ for final volume of 500 μl. This reaction occurred in 50 mM Tris-HCl, 100 mM NaCl, pH 8,3, containing imidazol 150 mM. The hydrolysis of S-2238 40 μM through thrombin formed by prothrombin activation by Lopap using 90 nM of the factor II or 90 nM of purified thrombin was evaluated pectrophotometrically in 405 nm during 10 minutes at 37° C.

### Description 6

Factor X Activating Activity:

Factor X (30 nM) was pre-incubated using Lopap 75 nM during 20 minutes at 37° C. in 120 μl of 25 mM Tris-HCl buffer pH 8,3 containing 200 mM NaCl and 10 mM CaCl₂. After that, 150 μl of 50 mM Tris-HCl buffer pH 8,3 containing 150 mM imidazol, 100 mM NaCl and 165 μl of 10 mM Tris-HCl buffer pH 8,0 containing 10 mM Hepes, 500 mM NaCl and 0,1% PEG 6000 were added up to the final volume of 500 μl. The formation of factor Xa was evaluated through the absorbency in 405 nm during 10 minutes at 37° C. after adding 150 μM of the substrate S-2765. The hydrolysis of 150 μM of the substrate S-2765 by 30 nM of the purified Factor Xa was examined using the experimental conditions described.

### Description 7

Lopap Activity Concerning the Purified Fibrinogen

Lopap (2 μM) was incubated both with and without factor II (90 nM) in 50 mM Tris-HCl buffer, containing 5 mM CaCl₂ and 100 mM NaCl, in a final volume of 300 μl during 10 minutes at 37° C. After that, purified human fibrinogen (7,5 μM) (Chromogenix) was added and the transformation of prothrombin into thrombin was evaluated through its coagulation time.

### Description 8

Experiment Concerning Enzyme Activity via Fluorogenic Substrate and Determination of Cleavage Sites:

The experiment was conducted using the quenched fluorescence substrate Abz-YQTFFNPRTFGSQ-EDDnp in a spectrofluorimeter Hitachi F-2000 on wavelength of 320 nm (activation) and 420 nm (emission). Before adding 10 μl of a storage solution of the substrate (prepared in DMF: H₂O, 1:1 v/v), the enzyme (73,3 pM) was incubated in a thermo-stable sterilizing recipient using 1,5 ml of 50 mM Tris-HCl buffer, pH 8,0 at 37° C. The kinetic constants Km and Kcat were determined from the data obtained by continuously measuring velocity during 10 minutes. The kinetic constants, with respective standard errors, were obtained through the Michaelis-Menten equation using the method described by Wilkinson. For determining the cleavage site, the peptidic fragments were separated through HPLC reverse-phase chromatography using a C₁₈ column. The elution solvents are TFA 0,1% in H₂O (solvent A), and acetonitrile-solvent A (9:1) as solvent B. The gradient used for separation was 10-100% of solvent B, with 1 ml/min flow. The cleavage sites were determined using the internal fragments of synthetic peptides as a standard.

### Description 9

Lopap Inhibition

The experiment for verifying Lopap enzyme aspects was followed through chromogenic substrates using inhibitors: PMSF (10 Mm) or E-64 (3.2 mM) incubated with Lopap (75 nM) final volume of 500 μl. The inhibitors were pre-incubated with Lopap during 15 minutes at 37° C. before adding the substrate S-2238 (40 μM).

### Description 10

The Influence of Bivalent Ions Concerning the Lopap Activity:

Lopap was exhaustively dialyzed against 100 mM EDTA during 48 h at 4° C. Lopap (75 nM), whether dialyzed or not, was incubated in presence or absence of CaCl₂ (5 mM), MgCl₂ (5 mM), or ZnCl₂ (5 mM), at 37° C. during 10 minutes. Then Factor II (90 mM) was added, and 40 μM of the chromogenic substrate S-2238, and 50 mM Tris-HCl buffer containing 100 mM NaCl, pH 8,3, in a final volume of 500 μl. The substrate hydrolysis was monitored spectrophotometrically at 405 nm during 20 minutes in the Beckman DU-7 equipment.

### Description 11

Titration of Lopap Serine Protease Activity Through NPGB

The experiment for the titration of the Lopap active site was conducted using the NPGB reagent, in accordance with the protocol previously described. The concentration of the active Lopap was determined through the titration using p-nitrophenyl-p′-guanidinebenzoate 0,47 μM (NPGB) in 0,1M Sodium barbital buffer, pH 8,3 at 37° C., in final volume of 1,0 ml. The p-nitrophenol resulted was quantified in absorbency using 410 nm in a Hitachi U-2000 spectrophotometer.

### Description 12

Determining the Prothrombin Fragments Induced by Lopap:

Lopap (30 nM) was incubated with prothrombin (500 nM) during 0, 1, 3, 6, 8 and 24 h at 37° C. in 500 μl of 50 mM Tris-HCl buffer, containing CaCl₂ (5 mM) and NaCl (100 mM) pH 8,0. The hydrolysis fragments resulted were analyzed through SDS-PAGE (10% gel) under reducing and non-reducing conditions and it was stained by the method of Coomassie Brilliant Blue R-250.

### Description 13

Purifying the Prothrombin Activator (Lopap):

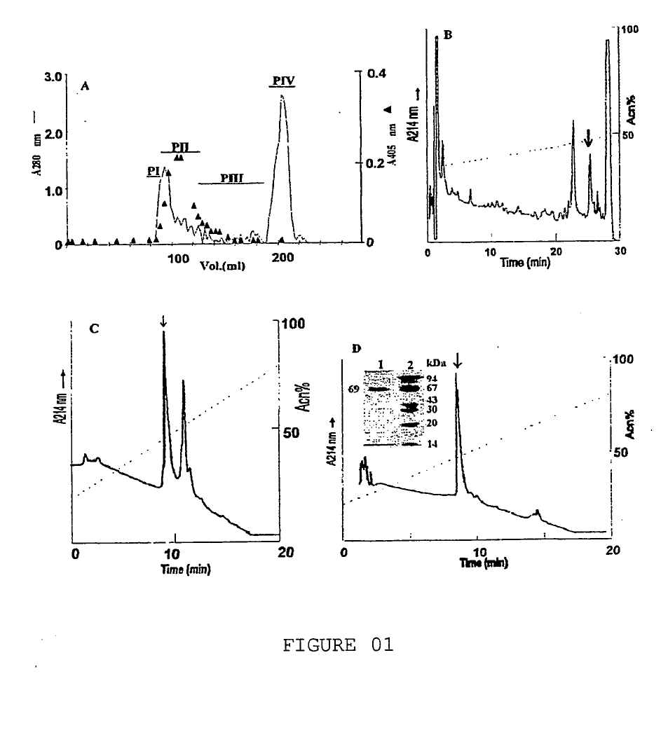

The Lopap purification process included a gel-filtration chromatography and two reverse-phase chromatographies. The protein profile obtained through the gel filtration chromatography is represented in FIG. 1 A. Only the PII peak has shown prothrombin activation capacity, which was submitted to the reverse-phase chromatography, resulting in peaks here represented in FIG. 1B. The prothrombin activating activity was detected in the eluted peak using 43% of acetonitrile (FIG. 1B). This activity fraction was submitted to a second reverse-phase chromatography resulting in two peaks, however only one of them showed prothrombin activating capacity (FIG. 1C). The active fraction was submitted to another reverse-phase chromatography using the same conditions, to confirm the presence of only one peak (FIG. 1D). This purification resulted in a protein, which maintains around 50% of activity, as can be seen in chart 1. The homogeneity. of the protein preparation is represented in FIG. 1D. The purified material showed a single band protein of approximately 69 KDa analyzed by SDS-PAGE.

### Description 14

Determination of the N-Terminal Sequence and Internal Peptides Sequence of Lopap

The N-terminal portion with 46 residues of amino acids (DVVIDGACPDMKAVSKFDMNAYQGTWYEIKKFPVANEANGDCGSVE) was obtained from purified Lopap, as well as the sequence of some internal peptides fragments called Fragments I: KSHVYTVPFGA. Fragment II: KSNQHRVNIWILSRTK Fragment III: VRAGHVE and Fragment IV: FDQSKFVETDFSEKACFF. The sequence that was obtained corresponded to about 15% of the whole protein considering 69 kDa its molecular mass.

### Description 15

Prothrombin Activating Activity by Lopap

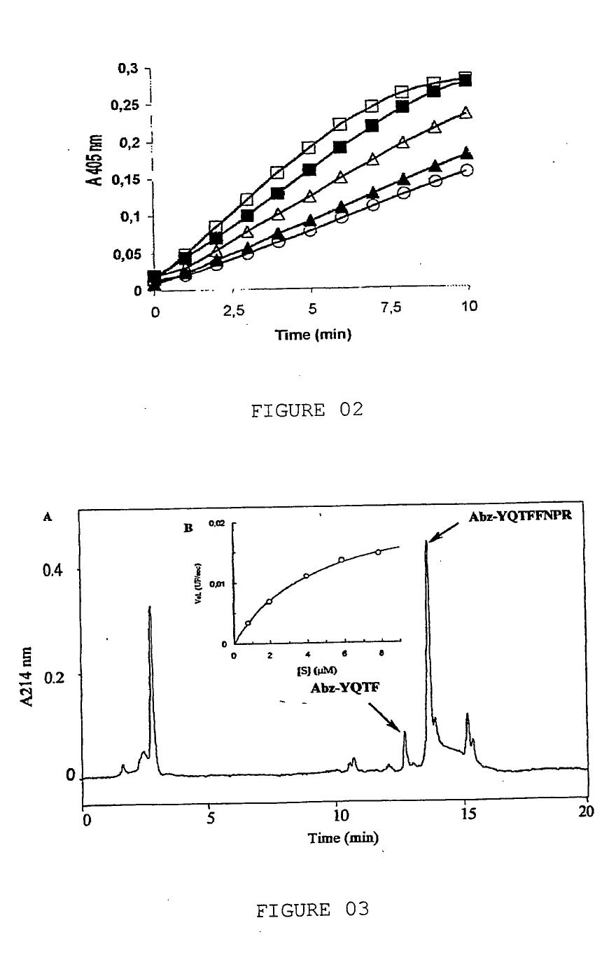

The thrombin produced from Lopap action on prothrombin occurred as dose-dependent manner (FIG. 2). Prothrombin (90 nM) was incubated with 75 nM of Lopap producing the same quantity of thrombin capable to hydrolyze the S-2238 substrate (40 mM), as well as the induced hydrolysis using 90 nM of purified thrombin. The thrombin activity was detected from 1 minute of pre-incubation.

### Description 16

Lopap Capacity of Activating Factor X:

Lopap did not present capacity of activating factor X and, besides, it was not capable to hydrolyze the S-2765 chromogenic substrate. The hydrolysis obtained using 75 nM of Lopap incubated during 10 minutes at 37° C. with 150 μM S-2765 substrate was of 0,34 μM. The concentration of p-nitroaniline formed during the reaction was calculated using the calorimetric determination with 8900 M⁻¹ cm⁻¹ as extinction coefficient at 405 nm. When Factor X (30 nM) was added to the experiment, the substrate hydrolysis obtained was of 2,6 μM. When Lopap was not used, the absorbency of the purified Factor Xa (30 nM) was of 34 μM.

### Description 17

Fibrinogen Coagulation by Lopap:

Lopap did not present activity like thrombin on purified fibrinogen, even after long time incubation (chart 2). However, a solid clot is formed after 240s when prothrombin is present. Ca²+addition has reduced the coagulation time to 60s.

### Description 18

Lopap Hydrolytic Activity on the Fluorogenic Peptide:

The kinetic parameters determined for Lopap using the quenched fluorogenic substrate Abz-QTFFNPRTFGSQ-EDDnp, based on the prothrombin sequence were Kₘₐₚₚ 4,5 μM; K_{cat} 5,32 sec^{-1;} K_{cat}/K ₘₐₚₚ 1,2×106 M^{-1 sec -1}. This indicates good relation and high catalytic efficiency for the studied enzyme, being these parameters obtained in accordance with what was described by Chagas et al. Lopap has shown activity on the Abz-YQTFFNPRTFGSQ-EDDnp substrate (deduced from prothrombin molecule) which was hydrolyzed in two sites Phe-Phe (10%) and Arg-Thr (90%) (FIG. 3)

### Description 19

Lopap Activity in Presence of Bivalent Ions

The Lopap activity was significantly decreased after the dialysis against EDTA, and can be substantially recovered when Ca²⁺ are added (FIG. 4). Besides that, the Lopap activity was completely annulled by 10 mM PMSF, while 3,2 mM E-64 did not affected it. The titration of the putative serines of Lopap by NPGB indicated the stechiometry of 1,2 serine residues by molecule of NPGB.

It can be seen in FIG. 4 that Lopap has shown augmented prothrombin activator activity after adding Ca²⁺ ions regardless to their activity in Calcium absence. After being exhaustively exposed to dialysis against EDTA, Lopap activity decrease about 75%, and may be gradually recovered through addition of rising concentrations of Ca²⁺ ions. Other bivalent ions, such as Mg and Zn²⁺ did not produce the same effect.

### Description 20

Determination of the Prothrombin Fragments Induced by Lopap:

On non reducing conditions, prothrombin hydrolysis (72 kDa) through Lopap resulted in several fragments (molecular mass of 52 kDa, of 36 kDa, of 27 kDa and of 16 kDa representing peptide F1/F2, prethrombin 2 or α-thrombin, Fragment-1 (F1) and Fragment-2 (F2) respectively. On reducing conditions, the prothrombin activation resulted in fragments with molecular mass of 52 kDa, of 36 kDa, of 32 kDa, of 27 kDa and of 16 kDa, representing F1/F2-activation peptide, prethrombin 2, thrombin B-chain, Fragment-1 (F1) and Fragment-2 (F2), respectively (FIG. 5)

### Description 21

Determination of the Prothrombin Activation Activity of Fraction II:

Pre-incubation of 15 to 300 nM of the purified fraction during 10 minutes at 37° C. with 90 pM of prothrombin with adding of 5 mM of CaCl₂ for final volume of 500 μL using 50 mM Tris-HCl, 100 mM NaCl, pH 8 as well as 150 mM of imidazol, with adding of 40 μM of the chromogenic substrate S-2238 (H-D-phenylalanyl-L-pipeaklyl-L-arginine-p-nitroanilide dihydrochloride) to the incubation mixture and evaluating spectrophotometrically the chromogenic substrate hydrolysis through 405 nm during 10 minutes.

### Description 22

Lopap Activity on a Normal Human Plasma:

For testing the procoagulant activity of Lopap, bristles crude extract (10 to 30 μg) or the purified enzyme (Lopap), 1 to 16 μg was incubated at 37° C. with 100 μl of normal human plasma. The pro-coagulant activity was evaluated after 6,25 mM of CaCl₂ addition through the coagulation time, with final volume of 400 μl. The plasma new calcification time, in presence of Lopap, was compared to the coagulation time of the plasma in absence of Lopap or. of crude extract (control).

### Description 23

The Effects of Lopap in the Microcirculatory System

Intravital Microscopic Studies:

The effects of Lopap in the microcirculatory system were determined in situ at the internal spermatic fascia of anesthetized (250 g Intraperitoneal sodium pentobarbital, 50 mg/kg.) rats. The surgery technique used for this procedure was described.

Briefly, the animals were maintained on a special board thermostatically controlled at 37° C., which included a transparent platform on which the tissue was placed to be transilluminated. The preparation was maintained humid and warm through irrigation of the tissue using Ringer-Locke warmed-up solution, 154 mM NaCl, 5,6 mM KCl, 2 mM CaCl₂, 6 mM NaHCO₃, and 6 mM of glucose, pH 7,2-7,4, containing 1% of gelatin. Through a color video camera accomplished to a triocular microscope (Axioskope, Carl Zeiss), microcirculation images were simultaneously visualized by TV monitors and computer images. The TV monitor images were recorded in video and the computer images were evaluated using software technology (KS300, Kontron) The images were obtained using a ×10/025 longitudinal distance objective/numeric aperture and ×1.6 otpovar. Lopap (100 μg/kg) was injected i.v. (Caudal vein) and the vessel microcirculation dynamics were observed through the monitors. The control animals received equivalent quantities of sterile saline. An hour after the injection and observing microcirculation, blood was collected from the abdominal aorta (500 μg) and blood coagulation time was measured.

### Description 24

In Vivo Study:

Lopap (100 μg/kg) was injected via caudal vein in male Wistar rats weighing from 200 to 250 g. Control rats received 150 mM of NaCl under the same conditions. After one hour of analysis their blood was collected through their abdominal aorta using disposable syringes. Blood for cell counting was collected in 2,7 mM of Na₂-EDTA, and for platelet aggregation studies in 139 mM of trisodium citrate (1 part for 9 parts of whole blood). Platelet poor plasma was obtained from citrate blood through centrifugation at 1900 g by 15 min. at 4° C. The platelet aggregation of the whole blood was performed as described in Sano-Martins IS, Santoro M L, Castro SCB, Fan H W, Cardoso JLC, theakson RDG. Platelet aggregation in patient's blood bitten by the Brazilian snake Bothrops jararaca. Thromb. Res. 1997; 87 (2): 183-95. Collagen (5 μg /ml. of final concentration) (Hormon-Chemie, Germany) was used as agonist for inducing platelet aggregation. For blood cell counting, Serono-Baker 9020+AX system was used, and. the fibrinogen was measured in accordance with von Clauss (gerinnungsphysiologische schnellmethode zur bestimmung des fibrinogens. Acta Haematol 1957, 17: 237-46) using reagents and controlling substances from Diagnostica Stago.

### Description 25

Histopathology:

The same animals of the in vivo studies were used for the histopathologic analyses. Brain, lungs, liver and kidneys fragments were collected and exposed for 48 hours to a solution containing 10% of formalin. Then they were soaked in paraffin and prepared for routine histology analyses and evaluated after staining them with eosin.

### Description 26

Statistics Analyses:

Student's T-test was applied through the statistics software Stata™ 5.0 in order to compare the platelet counting and the whole blood platelet aggregation in Lopap injected rats as well as in the blood of control rats.

Results:

a) Lopap activity on the plasma:

The bristle crude extract was incubated using citrate normal human plasma and the coagulation time obtained was between 290-80s (chart 1), while Lopap (1-16 μg) citrate normal human plasma coagulation has showed to require a similar time (chart 1)

b) Biological Tests using Lopap:
1. Intravital Microscopy Studies:

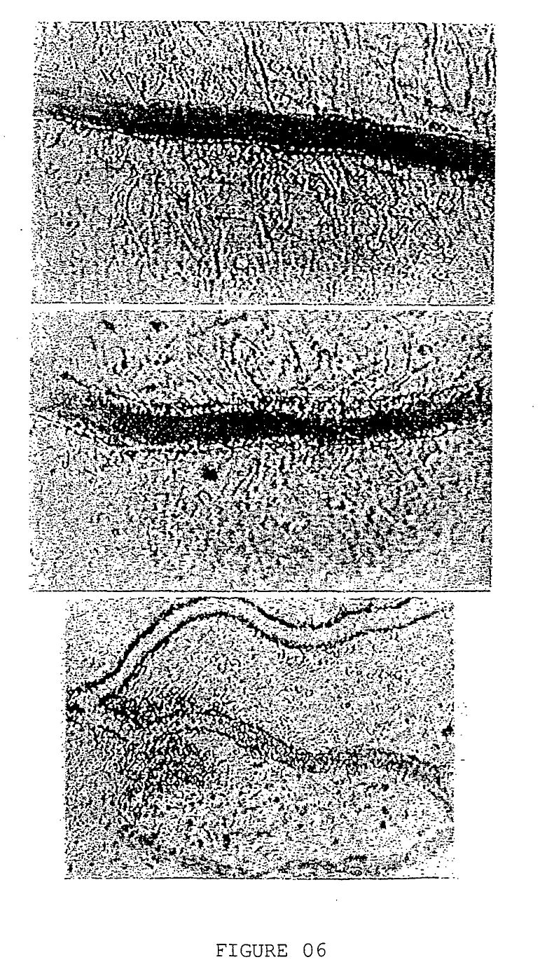

The protein intravenous administration provoked prominent alterations in the cremaster muscle microcirculatory system. Thrombus formation was observed in small vessels (10-30 μm of diameter), mainly in venules 5 minutes after injection. This effect was more evident after 40 minutes when systemic envenomation with total venular stasis and thrombus at arteriolar vessels were clearly visualized (FIG. 6). Haemorragical areas were visualized 30 min. after administering Lopap. One hour after the injection, blood collected from the animals treated with Lopap was not unclotable. Control animals treated with saline solution did not present microcirculatory alterations.
2. Coagulation Parameters “In-Vivo”:

The platelet counting evenly decreased in about 40% in Lopap injected rats, when compared to those of the control rats. The collagen induced platelet aggregation was annulled in the blood of envenomed rats. No morphological or quantitative alteration both in erythrocytes and in leukocyte cells was observed. No fibrinogen was detected in these animals' plasma.
3. Histopathology:

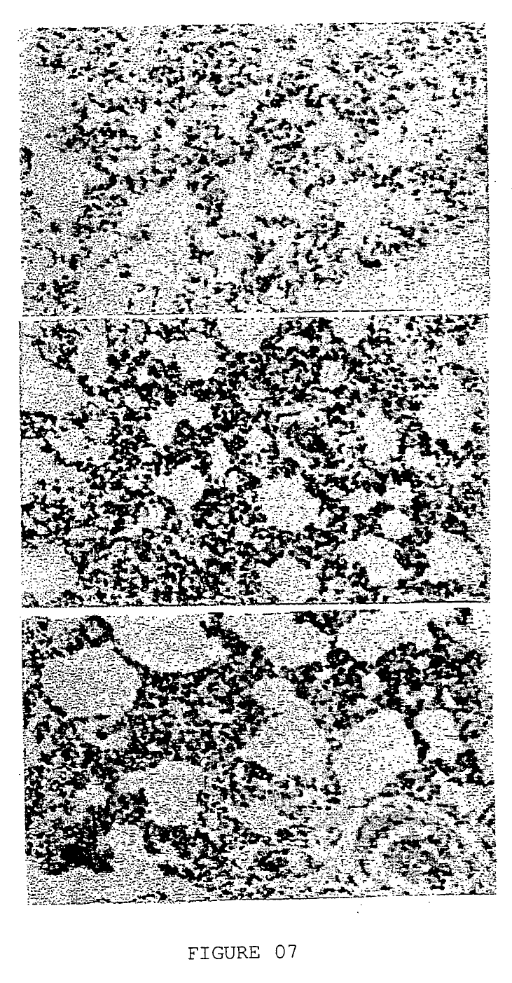

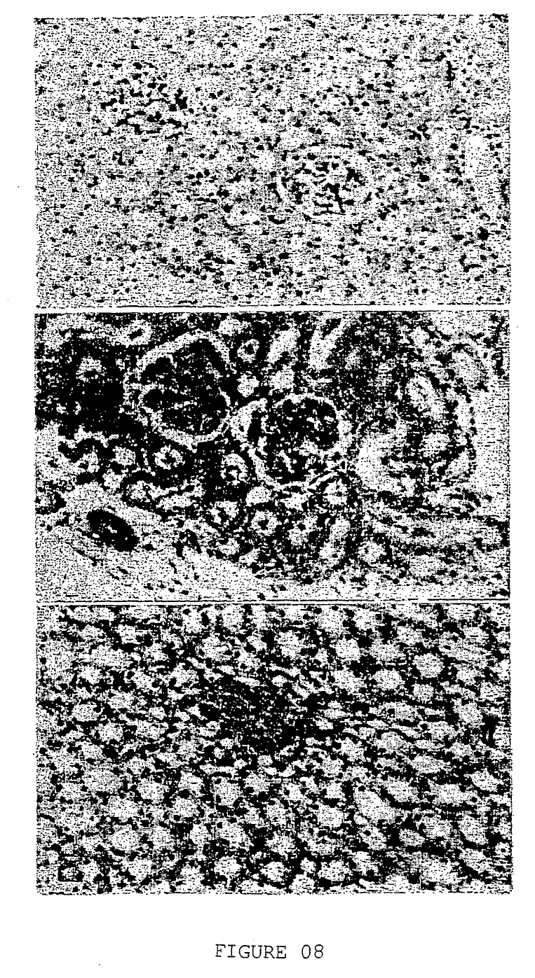

One hour after the Lopap injection, a significant leukocyte infiltration was observed in the lungs of the experiment animals. (FIG. 7B and C). Neutrophiles and monocytes adhered to the endothelial cells of small blood vessels. These cells were also detected in the organ parenchyma spaces (FIG. 7C). A significant vascular congestion was observed in glomerular vessels and in vessels between the proximal and distal renal tubules (FIG. 8b). The hemorrhage was not only observed in glomerular vessels but also in other vessels of the organ. Concerning the medullar area, tubule cells have showed focal areas of hyaline necrosis. Histology alterations were not found when other organs were analyzed.

Accidental contacts with the bristles of the Lonomia obliqua caterpillar cause unclotability and alterations in the coagulation factors related to the thrombin and can result in hemorrhagic syndrome. Pro-coagulating proteins such as factor X and the prothrombin activators of animal venom are responsible for the consumption coagulopathy through the fibrinogen depletion. Although the most important way of activating prothrombin is through the prothrombinase complex, prothrombin can also be activated by exogenous factors, such as snake venom components through different manners.

After comparing the prothrombin hydrolysis products generated by Lopap (chart 3) with the fragments produced by other prothrombin activators, a mechanism of action may be suggested involving the formation of prethrombin 2 and thrombin.

Since apparently the meizothrombin is not formed by Lopap and, products with molecular mass similar to the prethrombin 2 are produced, Lopap could be up be classified as a Type 4 activator. However, activators of Type 4 are not able to convert prothrombin in active enzyme products while Lopap is able to produce active thrombin. On the other hand, the molecular mass of the fragments that were formed is similar to those formed by factor Xa, when in presence of prothrombinase complex. Besides that, the results obtained from the hydrolysis of the quenched fluorescence substrate have shown that the cleavage in the main chain occurs in the same cleavage bound as by thrombin (Arg-Thr).

Self-catalysis is one of the main problems detected when performing the hydrolysis experiment involving prothrombin and the real Lopap activation mechanism on the prothrombin. It may only be elucidated and confirmed when a recombinant prothrombin could be.used and also the mass spectrometry analysis and the amino acids sequence of the fragments are performed.

From bristles extract of the L. obliqua, the authors of the herein invention purified a prothrombin activator serine protease of 69 kDa. The preliminary results have shown that the Lopap activating capacity is independent of the prothrombinase complex, however the Ca²⁺ ions provoke an increase of this activity. The Lopap purifying process included the use of organic solvents, causing a visible activity loss up to about 50% when using 30% of acetonitrile and 80% when using 50% of acetonitrile (chart 1), therefore it is quite difficult to calculate the protein specific activity. Less radical purifying methods were not so efficient and currently the production of recombinant Lopap is being performed.

Lopap was characterized as being a serine protease activated through Ca²⁺ ions and it is structurally different from other prothrombin activators described in literature. The N-terminal segment showed 45,6% of identity when compared to the N-terminal portion of the purified insecticyanin of the Manduca Sexta hemolymph.

Fragments I, II, III and IV showed respectively 36,4%, 37,5%, 42,9% and 55,5% of identity with the internal fragments sequence of the same protein (G ₈₆-Q₉₇; D₁₂₄-E₁₄₈; I₁₆₀-Y₁₇₇).

The homogeneity of purified Lopap was confirmed through only one N-terminal residue. The quenched fluorescence substrate was programmed for containing the thrombin bound Arg₂₈₄-Thr₂₈₅, flanked by the sequence Tyr₂₇₇-Ser₂₈₈. Lopap cleaved this substrate in the peptidic bound corresponding to prothrombin cleaved by thrombin.

It was demonstrated by the herein invention that Lopap is not able to activate the factor X, and, differently than Lopap, the activator of the Factor X will require to be purified (preliminary results) from crude extract of the L. obliqua bristles. There are at least two procoagulant components in such venom (chart 3).

According to this invention, Lopap is a new prothrombin activator, what comes to be a quite important factor responsible for consumption coagulopathy found in patients exposed to the venom of the L. obliqua caterpillar.

The purified protein in low doses, by its capacity of activating prothrombin-generating thrombin, withdraws fibrinogen from circulation under controlling conditions, transforming it into fibrin microclots. The decrease of the plasmatic fibrinogen concentration allows that blood coagulation time lasts longer avoiding severe vascular thrombosis.

Since Lopap does not present fibrinogenolytic activity, the coagulating capacity of the fibrinogen not consumed in the process could be preserved. This way, the fibrinogen plasmatic concentration would be decreased, however patients would not have predisposition for hemorrhagic state. Besides that, it could be used for preparing diagnosis KITS for detecting plasmatic prothrombin in dysprothrombinemias (Kini R M, Rao V S, Joseph J S. Haemostasis; 2001. p 218-24).

CHART 1
The influence of the acetonitrile in the Lopap
(300 nM) activity was tested using different
concentrations of acetonitrile. Its activity was
indirectly determined through the thrombin
formation experiment from the prothrombin using
the chromogenic substrate S-2238.

| Acetonitrile | | | | Hydrolysis |
|---|---|---|---|---|
| (%) | Lopap | F II | S-2238 | (%) |
| 0 | + | − | + | 0 |
| 0 | − | + | + | 0 |
| 0 | + | + | + | 100 |
| 30 | + | + | + | 50.7 |
| 50 | + | + | + | 21.7 |
| 90 | + | + | + | 1.8 |

CHART 2
Fibrinogen coagulation through Lopap.
Lopap (2 μM) was incubated during 10 min. at 37° C.
using or not Factor II (90 nM), in 50 mM Tris-HCl
buffer containing 5 mM CaCl₂ and 100 mM NaCl in a
final volume of 300 μl. Purified human fibrinogen
(7.5 μM) was added and the transformation of
prothrombin into thrombin was evaluated through
the coagulation time of the fibrin FG = fibrinogen.

| | | | | | Coagulation |
|---|---|---|---|---|---|
| Lopap | FXa | F II | Ca²⁺ | FG | Time (S) |
| − | + | + | + | + | 120 |
| − | − | + | − | + | >1200 |
| − | − | + | + | + | >1200 |
| + | − | − | − | + | >1200 |
| + | − | + | + | + | 60 |
| + | − | + | − | + | 240 |

CHART 3
Comparing the prothrombin fragments obtained after
the hydrolysis with different activators, analyzed
by SDS-PAGE. A: reducing conditions; B: non-reducing conditions.

| | | | | O. | | | |
|---|---|---|---|---|---|---|---|
| | Molecular | | | scutellatus | | | |
| | Mass | Ecarin | | Activator | | Lopap | |
| Fragment | (kD) | A | B | A | B | A | B |
| Prothrombin | 72 | + | + | + | + | + | + |
| Meizothrombin | 72 | − | + | − | + | − | − |
| F1/F2/A | 55 | + | + | + | − | − | − |
| chain | | | | | | | |
| F1/F2 chain | 52 | + | + | − | + | + | + |
| α-thrombin | 36 | − | + | + | + | − | + |
| Prethrombin 2 | 36 | − | − | + | + | + | + |
| Frag. B of | 32 | + | − | + | − | + | − |
| thrombin | | | | | | | |
| Fragment 1 | 27 | + | + | + | + | + | + |
| Fragment 2 | 16 | + | + | + | + | + | + |

Purification of the Prothrombin Activator Lopap Found in the Bristles Extract of the Lonomia Obliqua Caterpillar.

A) Gel-filtration chromatography in Sephadex G-75. The capacity of prothrombin activation was detected using chromogenic substrate S-2238.

B) Reverse phase chromatography (HPLC System, column C4) of the fraction PII of the gel-filtration stage after elution with a linear gradient of 35-50% of B solvent.

C)Second reverse-phase chromatography as previously described, except that, in this case the gradient used was of 20-80% of solvent B.

D)Reverse-phase chromatography of the peak PII-4R2 as previously described. Detail: SDS_PAGE of 20 μg of the purified protein (line 1), and molecular mass standard (line 2): phosphoripase B, 94 kDa; albumin, 67 kDa, ovalbumine, 43 kDa; carbonic anidrase, 30 kDa; trypsin inhibitor, 21 kDa; α-lactoalbumin, 14.4 kDa.

FIG. 2:

Lopap (15-300 nM) was pre-incubated during 10 min. at 37° C. with prothrombin 90 nM and incubated at 37° C. using the chromogenic substrate S-2238 (40 μM) exposed to 5 mM CaCl₂ in the final volume of 500 μl.

◯ 15 nM; ▴ 30 nM; Δ 75 nM; ▪ 150 nM; □ 300 nM.

FIG. 3:

Hydrolysis of the Fluorogenic Substrate Through Lopap:

A) Abz-YQTFFNPRTFGSQ-EDDnp was incubated with Lopap in 50 mM Tris-HCl buffer, pH 8,0 at 37° C. for 3 h. The incubation mixture was analyzed through chromatography in HPLC as described in Manner and Process of making and using it.

B)The Michaelis-Menten profile obtained with 0.8-8.0 μM of fluorescent substrate hydrolysed by 73.3 pM of Lopap.

FIG. 4:

The Influence of Bivalent Ions in the Lopap Capacity of Prothrombin Activation.

Lopap (75 nM), whether dialyzed or not, was incubated at 37° C. with 40 μM of chromogenic substrate S-2238 and prothrombin 90 nM; ● Control: without prothrombin; □ reaction with non dialyzed Lopap using 5 mM CaCl₂; ▪ reaction with non dialyzed Lopap without using Ca²⁺; ▴ Dialyzed Lopap against 100 mM EDTA; Δ reaction using dialyzed Lopap using 5 Mm CaCl₂; ★ reaction using dialyzed Lopap using 5 mM MgCl₂; ◯ reaction using dialyzed Lopap using 5 mM ZnCl₂.

FIG. 5:

SDS-Page Profile of Prothrombin Hydrolysis Through Lopap:

Profile of prothrombin hydrolysis through Lopap. Human prothrombin (500 nM) incubated with Lopap (30 nM) during 0.1.3.6.8 and 24 h and analyzed on SDS-PAGE (polyacrilamide gel of 10%) after reduction conditions. Controls: FII (human prothrombin) and Factor IIa (human thrombin, 12 U).

1. Purifying process of soluble proteins of the L. obliqua bristles through prothrombin activation, characterized for containing the following stages:
a) Homogenize L. obliqua bristles in phosphate-buffered saline (PBS), pH 7.4-8.0, centrifuge at 4° to 10° C. by 2500×g from 30 to 60 minutes to obtain a crude extract from the prothrombin activator;
b) Purify the prothrombin activator from 50 to 200 mg of whole protein from 2 to 10 ml of crude extract through gel-filtration chromatography in Sephadex G-75 resin. Elute in 20 to 50 mM Tris-HCL buffer containing NaCL 50 to 100 mM and benzamidine 2 to 5 mM, pH 7.4 to 8.0 with flow of 1,0 ml/h;
c) Collect fractions from 1 to 3 ml and monitor the chromatography protein profile by UV absorbency in 280 nm;
d) Activate the prothrombin using the protein peaks obtained and the S-2238 calorimetric substrate, specific for thrombin;
e) Obtain the peak PII presenting the activation of prothrombin;
f) Submit the active fraction obtained to a reverse-phase chromatography in column C4 using HPLC analytic system. Use as solvents: —A: 0,1% TFA in water (balanced) and B: solvent A and acetonitrile in a proportion of 1:9 (elution) and proceed the protein detection of 214 to 280 nm in UV monitor;
g) Collect fractions of 0.5-1.0 ml and lyophilize them immediately for eliminating acetonitrile;
h) Suspend again the lyophilized samples in 20 to 50 mM Tris-HCL buffer containing 50 to 100 mM NaCL, pH 7.4 to 8.0;
i) Test activation of prothrombin activator of the fractions as described in item d);
j) The active peak in fractions is eluted between 42 to 44% of solvent B;
k) Submit the active fraction again to a chromatography as described in item (f) using a gradient between 20-80% of solvent B, during 20 minutes;
l) Repeat the stages from (f) through (j);
m) Submit the purified material to an electrophoresis in polyacrilamide gel containing SDS for homogeneity evaluation. This gel could be stained by Coomassie brilliant blue;
n) Evaluate the final protein concentration by protein assay using colorimetric methods or Absorbency in 280 nm in order to obtain the prothrombin activator;

2. Process in accordance with claim 1, characterized by using in stage (b) the following solvents for elution:
solvent A: 0,1% TFA in water and solvent B: solvent A and acetonitrile in a proportion of 1:9.

3. Process in accordance with claim 1 characterized by using the HPLC analytic system in stage (f) produced by Merck-Hitachi (D-2500 model) and the monitor of stage (g) produced by Shimadzu UV (SPD-6AV model);

4. Process in accordance with claim 1 characterized by using the HPLC purification in the stage (f) using a gradient of 35-50% of solvent B;

5. Process for partial determination of the amino acids sequence of the prothrombin activator characterized by degrading 500-1000 pM of purified protein with 10 pmol of trypsin in 100 mM Tris-HCl, pH 8.0 containing 0.02% of CaCl₂ during 18 hours at 37° C. stopping the reaction with 15% (v/v) of formic acid;

6. Process in accordance with claim 5 characterized by separating through HPLC, the fragments obtained in the column C4, eluted with solvents 0,1% of TFA in water (solvent A) and acetonitrile: solvent A (9:1) (solvent B);

7. Process in accordance with claim 6 characterized by using a gradient of 0-100% of solvent B with flow of 1.0 ml/min during 30 min for the HPLC separation;

8. Process in accordance with claim 7 characterized by determining sequence of four internal peptides and the N-terminal sequence;

9. Process in accordance with claim 8 characterized by N-terminal portion containing 46 residues of amino acids (DVVIDGACPDMKAVSKFDMNAYQGTWYEIKKFPVANEANGDCGSVE) (SEQ ID NO: 1) and the internal peptides fragments being:
Fragments I (KSHVYTVPFGA) (SEQ ID NO: 2); Fragment II (KSNQHRVNIWILSRTK) (SEQ ID NO: 3); Fragment III (VRAGHVE) (SEQ ID NO: 4) and Fragment IV (FDQSKFVETDFSEKACFF) (SEQ ID NO: 5);

10. Process in accordance with claim 9, characterized by the sequence obtained of about 15% of the whole protein considering 69 KDa its molecular mass;

11. Process for determination of the prothrombin activation of fractiion II, characterized by containing the following stages:
a) Pre-incubate 15 to 300 nM of the purified fraction during 10 minutes at 37° C. with 90 pM of prothrombin using 5 mM of CaCl₂ for final volume of 500 μL using 50 mM Tris-HCl, 100 mM NaCl, pH 8 as well as 150 mM of imidazol;
b) Add 40 μM of chromogenic substrate S-2238 (H-D-phenylalanyl-L-pipicolyl-L-arginine-p-nitroanilide dihydrochloride), to the incubation mixture and evaluate spectrophotometrically the chromogenic substrate hydrolysis through 405 nm during 10 minutes;

12. N-terminal sequence and sequence of internal fragments of the prothrombin activator fraction characterized by containing 46 residues of amino acids (DVVIDGACPDMKAVSKFDMNAYQGTWYEIKKFPVANEANGDCGSVE) (SEQ ID NO: 1) in the N-terminal portion and the internal peptide fragments are:—Fragment I (KSHVYTVPFGA) (SEQ ID NO: 2); Fragment II (KSNQHRVNIWILSRTK) (SEQ ID NO: 3); Fragment III (VRAGHVE) (SEQ ID NO: 4) and Fragment IV (FDQSKFVETDFSEKACFF) (SEQ ID NO: 5) and the sequence obtained corresponds to about 15% of the whole protein with molecular mass of 69 KDa;

13. Prothrombin activator was obtained in accordance with the process of claims from 1 through 11, characterized by containing the following structure: The purified protein is characterized as a serine protease which hydrolyses the prothrombin generating Fragments 1, 2 and thrombin as showed in the figures;

14. The utilazation of the prothrombin activator of claim 13, characterized by enabling to be using prothrombin activator as a dysfibrinogening agent in prothrombotic state patients;

15. The utilization of the prothrombin activator of claim 13, characterized by enabling to be used for producing diagnosis kits for detecting plasmas prothrombin in hemmorhragic state patients.


SEQUENCE LISTING (7 sequences)

SEQ ID NO: 1 - protein, 46 aa, Lonomia obliqua
  ref <301>: AUTHORS: Chudzinski-Tavassi A.M.; Reis C.V.
  ref <302>: TITLE: A prothrombin activator serine protease from the Lonomia obliqua caterpillar venom (Lopap): biochemical characterization.
  ref <303>: JOURNAL: Thrombosis Research
  ref <304>: VOLUME: 102
  ref <305>: ISSUE: 5
  ref <306>: PAGES: 427-436
  ref <307>: DATE: 2001-06-01
DVVIDGACPDMKAVSKFDMNAYQGTWYEIKKFPVANEANGDCGSVE

SEQ ID NO: 2 - protein, 11 aa, Lonomia obliqua
KSHVYTVPFGA

SEQ ID NO: 3 - protein, 16 aa, Lonomia obliqua
KSNQHRVNIWILSRTK

SEQ ID NO: 4 - protein, 7 aa, Lonomia obliqua
VRAGHVE

SEQ ID NO: 5 - protein, 18 aa, Lonomia obliqua
FDQSKFVETDFSEKACFF

SEQ ID NO: 6 - protein, 12 aa, Artificial Sequence
  Synthetic fluorogenic substrate
QTFFNPRTFGSQ

SEQ ID NO: 7 - protein, 13 aa, Artificial Sequence
  Synthetic fluorogenic substrate
YQTFFNPRTFGSQ